(19) United States
(12) Patent Application Publication
(10) Pub. No.: US 2025/0185915 A1
(43) Pub. Date: Jun. 12, 2025

(54) Digitales Dermatoskop

(71) Applicant: Magnosco GmbH, Berlin (DE)

(72) Inventors: Axel Fett, Berlin (DE); Christoph Seifert, Eichwalde (DE); Goran Stankovic, Berlin (DE); Fabian Patrovsky, Berlin (DE)

(21) Appl. No.: 18/913,147

(22) Filed: Oct. 11, 2024

(30) Foreign Application Priority Data
Oct. 11, 2023 (EP) 23203017.1

(51) Int. Cl. A61B 5/00

(52) U.S. Cl. CPC A61B 5/0077; A61B 5/002; A61B 5/443; A61B 5/742; A61B 5/748; A61B 2560/0214

(57) ABSTRACT

A digital dermatoscope for skin examination, the digital dermatoscope being formed as a hand-held device, wherein the digital dermatoscope comprises a handle part which can be grasped by a hand and a head part adjoining the handle part and having an imaging unit, wherein the imaging unit has an imaging unit housing arranged on a lower side of the head part, an image sensor accommodated inside the imaging unit housing, and a lens, wherein an exchangeable spacer attachment contacting the skin to be examined during a skin examination is arranged at an end of the imaging unit housing, the digital dermatoscope further comprising a control unit and a first display arranged on an upper side of the head part, wherein the control unit is formed to drive the first display to display images of a skin section to be examined acquired by means of the imaging unit.

The invention relates to a digital dermatoscope for examining the skin of a patient.

Dermatoscopy, also known as dermoscopy or reflected-light microscopy, enables physicians to quickly visualise skin structures down to the papillary dermis. The skin patterns visible during dermatoscopy are mostly due to pigment and vascular structures.

Compared to examination with the naked eye, dermatoscopy enables better differentiation of (early) malignant melanomas (so-called black skin cancer) from its benign simulators. Dermatoscopy is given different names depending on the localisation and disease state: Trichoscopy is used for scalp and hair diseases, inflammoscopy for inflammatory diseases and entomodermoscopy for the examination of infestations and infectious states using dermatoscopy. In this way, the condition of the skin and its alterations can be observed and early detection of malignant tumours of the skin, such as malignant melanoma, can be achieved.

Until now, analogue dermatoscopes have mostly been used. These analogue dermatoscopes are usually hand-held magnifying glasses with appropriate magnification and built-in lighting to illuminate the examination area. For the purpose of skin examination, a first side of the head part of such an analogue dermatoscope is placed on the skin section to be examined, light is irradiated onto the skin section to be examined by means of the dermatoscope and reflected by the skin. The reflected light exits the analogue dermatoscope after passing through an optical system on the opposite side of the head part and thus reaches the eye of the person carrying out the examination.

Analogue dermatoscopes have a number of disadvantages. For example, recordings of the skin cannot be saved as images and therefore cannot be shown to the patient or used for long-term documentation. The person carrying out the examination can only describe the details of the examined skin section in words and record them as documentation. This makes long-term traceability particularly difficult and relatively imprecise. Furthermore, the skin can be magnified to a limited extent only using an analogue dermatoscope, in particular only optically and usually only with a fixed magnification factor.

In principle, it is possible to use an analogue dermatoscope together with a digital camera, such as a smartphone. For example, the camera lens of the smartphone could be placed on the head part of the analogue dermatoscope so that the light reflected from the skin does not fall into the eye of the person carrying out the examination, but into the camera lens. However, this requires the use of a second device, which is inconvenient. In addition, such digital cameras are not designed for skin examinations and, in particular, are not designed to work with the optics of the analogue dermatoscope. This can lead to problems with focusing the lens and/or to imaging errors. In addition, the use of smartphones without special technical solutions is particularly problematic in terms of data protection law, as unauthorised third parties may gain access to sensitive patient data.

Based on the state of the art described above, the task is to provide a dermatoscope which does not have the disadvantages mentioned and in particular enables a simpler examination of the skin and/or skin appendages and a more reliable diagnosis.

The invention solves the task with a dermatoscope according to claim 1. Advantageous embodiments are the subject matter of the dependent claims, the description and the figures.

The invention relates to a digital dermatoscope for skin examination, the digital dermatoscope being formed as a hand-held device, wherein the digital dermatoscope comprises a handle part which can be grasped by a hand and a head part adjoining the handle part and having an imaging unit, wherein the imaging unit has an imaging unit housing arranged on a lower side of the head part, an image sensor accommodated inside the imaging unit housing, and a lens, wherein an exchangeable spacer attachment contacting the skin to be examined during a skin examination is arranged at an end of the imaging unit housing, wherein the digital dermatoscope further comprises: a control unit and a first display arranged on an upper side of the head part, wherein the control unit is formed to drive the first display to display images of a skin section to be examined acquired by means of the imaging unit.

The dermatoscope according to the invention is digital, since it is formed to acquire digital images of the skin and/or skin appendages and to display these images on the first display and preferably store the images digitally. Hereinafter the skin examination using the dermatoscope according to the invention is discussed, whereby the examination of the nails or hair is also always included. Similar to the setup of an analogue dermatoscope, the digital dermatoscope has a handle part and a head part. The handle part can be grasped by an operator to carry out a skin examination with the digital dermatoscope. For skin examinations, the spacer attachment of the digital dermatoscope is placed on the section of a patient's skin to be examined so that the free end of the spacer attachment pointing away from the head part contacts the skin surface. The spacer attachment has an opening at the free end through which the light emitted by the digital dermatoscope can reach the skin. For this purpose, the digital dermatoscope can have an illumination unit preferably comprising annularly arranged LEDs (LED=light-emitting diode). In particular, the illumination unit can be a darkfield illumination unit. The light emitted by the digital dermatoscope is reflected by the illuminated section of the skin, picked up by the lens of the imaging unit and focused onto the image sensor. The image sensor generates a digital image from the reflected light, which the digital dermatoscope displays on the first display subsequently. The control unit is formed to drive the components of the digital dermatoscope required for this purpose. In other words, the control unit is formed to display the image acquired by the image sensor on the first display and, if necessary, to store it in a memory unit of the digital dermatoscope. The image of the skin section to be examined thus displayed on the first display can be viewed by the person carrying out the examination (hereinafter also referred to as the operator). The control unit can comprise a processor. The control unit can be formed as a “system-on-module” (SOM) or “system-on-chip” (SOC), i.e. as an integrated circuit. In particular, the SOM/SOC can be used in combination with a carrier board, which can be used to realise individual functions and/or interfaces of the digital dermatoscope. For example, the SOM/SOC can be connected to the carrier board through predefined pins. With such a setup, the required installation space can be reduced and the SOM/SOC can also be replaced, for example to retrofit a more powerful SOM/SOC at a later time.

The imaging unit is arranged in or on the head part of the digital dermatoscope. In particular, the imaging unit housing and thus the imaging unit is arranged on a lower side of the head part or extends downwards from the head part, the lower side here meaning the side of the head part facing the skin in the state of use. Thus, the imaging unit extends towards the skin. In particular, the imaging unit is integrated into the head part, preferably through the imaging unit housing. The imaging unit can, for example, be screwed or welded to the head part, preferably through the imaging unit housing. The image sensor can be, for example, an 8 MP to 12 MP sensor, preferably a 12 MP sensor, i.e. have a resolution of 12 megapixels, preferably 12 megapixels. This allows the skin to be examined at a sufficiently high resolution so that any skin diseases or dermatoses can be easily recognised. Of course, a higher resolution may also be possible. Preferably, the lens has a fixed focal length, for example a fixed focal length of 8 mm. The lens can have an aperture of f/4, for example. The imaging unit housing and thus the imaging unit is connected at a first end to the head part of the digital dermatoscope and at a second end opposite the first end to the spacer attachment.

The spacer attachment ensures a predefined distance between the imaging unit, in particular the lens, and the skin over its longitudinal extension along the optical axis for image acquisition. This is particularly necessary if the imaging unit of the dermatoscope can only focus at the fixed distance to the skin under examination due to a possible fixed focal length of the lens. In particular, the digital dermatoscope, or the lens thereof, can therefore have a fixed focal length. Fixed focal lengths have the particular advantage of providing a better resolution. The spacer attachment thus ensures that the section of skin to be examined is arranged on the focal plane of the imaging unit. For this purpose, the free end of the spacer attachment of the digital dermatoscope must be placed on the skin, i.e. in contact with the skin. The spacer attachment can specify an acquisition diameter (or, in other words, an image viewing diameter) for the image to be acquired by means of the diameter of the opening at the free end thereof; in particular, the opening is of circular shape.

The spacer attachment is replaceable and can be replaced by a spacer attachment with other image-influencing properties, such as a spacer attachment with a different opening diameter, in particular. Skin sections of different sizes can therefore be examined using different spacer attachments. For example, examinations on a relatively large area can be carried out using a spacer attachment with a large opening diameter and examinations on a relatively small area, such as skin examinations of narrow areas of the body, can be carried out using smaller spacer attachments. The spacer attachment is preferably attached to, or alternatively inserted into, the free end of the imaging unit housing. For this purpose, the imaging unit housing can have a recess, preferably circular, at its free end and the spacer attachment can have an endpiece shaped to correspond to the circular recess, so that, when the spacer attachment is attached to the imaging unit housing, the endpiece overlaps with the circular recess. However, the recess can also have a different shape, for example a rectangular, oval or star shape. The spacer attachment is preferably held magnetically to the imaging unit housing.

The first display is an electronic display, in particular an LCD display, for example with LED backlighting. The first display is arranged in the head part of the dermatoscope in such a way that it can be read from the upper side of the head part and thus of the digital dermatoscope. Here, the upper side refers to the side of the digital dermatoscope facing the operator during the examination. The first display can be covered by a cover glass, i.e. a cover glass can be arranged above the first display. The first display is preferably arranged on an optical axis of the imaging unit (in particular of the lens), in particular in such a way that the optical axis passes through the centre of the first display. This makes using the digital dermatoscope particularly intuitive, as the operator has the impression of “looking through the dermatoscope” in a straight line, as with the analogue dermatoscopes they are already familiar with. More preferably, the first display has a (circular) round shape, which also contributes to intuitive operation due to its similarity to an analogue dermatoscope. Skin diseases or dermatoses, or malignant melanomas can be examined effectively in this way. The first display can be formed as an image-only display, i.e. not as an operating element. However, the first display is preferably a touch display, i.e. it can be operated using one or more fingers of the operator. For example, the first display can be formed to recognise touch gestures performed by the operator using their fingers on the display surface and to perform an action in response to recognising a predefined touch gesture. Therefore, the display is preferably formed to recognise a touch gesture that triggers a zoom function. This allows the operator to enlarge or minimise, directly on the first display, an image of the skin section to be examined being displayed on the first display using a touch gesture, in particular by using a digital zoom. For example, a “pinch-to-zoom” gesture can be provided for this purpose, in which zooming out is achieved by moving two fingers towards each other and zooming in by moving two fingers away from each other. The first display adopts the “magnifying glass character” of an analogue dermatoscope and transfers it into the digital world for the operator.

As already mentioned, the digital dermatoscope according to the invention is formed to acquire digital images of the skin and to display these images on the first display. In particular, the digital dermatoscope can acquire multiple images per second and display them on the first display as a live view, i.e. as live video. Individual still images of the skin can also be acquired and displayed on the first display. The digital dermatoscope is preferably formed to store recordings of the skin as digital images, for example in a memory unit of the digital dermatoscope. More preferably, the digital dermatoscope, in particular the control unit, is formed to display the acquired image on the first display in false colours or in black and white. More preferably, the digital dermatoscope is formed to change a contrast value of the acquired image displayed on the first display.

The acquired images can be displayed on the first display not only during the examination, but also when the digital dermatoscope is removed from the skin. In particular, the digital dermatoscope can be formed to display images acquired during previous examinations as well. This means that the patient can be shown the examined skin sections directly on the first display without having to leave the dermatoscope on the skin. Long-term documentation of the examined skin section is also possible, for example by storing the images of multiple examinations in the digital dermatoscope itself or by transferring the images to another electronic device, such as a computer, a tablet or a smartphone. The images can be shared with another physician or analysed automatically, for example using artificial intelligence, for the purpose of diagnosis. In addition, depending on the resolution of the image, individual portions of an image acquired using the digital dermatoscope can be enlarged during the examination, for example using a digital zoom, or also after the examination. The digital dermatoscope is therefore not set to a component-related fixed magnification factor. The digital dermatoscope has a particularly compact and simple design, as the imaging unit is arranged in the head part of the digital dermatoscope, i.e. it is integrated into the digital dermatoscope. In addition, if the control unit is formed accordingly, the acquired image can be displayed on the first display in a modified form, for example in false colours or with altered contrast, which can make it easier for an operator to recognise skin diseases or dermatoses. The digital dermatoscope according to the invention enables a simpler, more user-friendly examination of the skin as well as a more reliable diagnosis.

According to one embodiment, the first display has a shape corresponding to the shape of the head part. According to one embodiment hereof, the head part and/or the first display have a (circular) round shape. In particular, the first display and the head part have corresponding (circular) round shapes. As already mentioned, a round first display contributes to intuitive operation due to its similarity to an analogue dermatoscope and also enables a particularly effective skin examination. In particular, this enables a particularly effective examination of skin diseases or dermatoses, such as malignant melanomas, which are at least approximately round in shape. In particular, a round display adopts the “magnifying glass character”, translating the analogue dermatoscope into the digital world for the user. A further similarity to an analogue dermatoscope is achieved by the correspondingly shaped (round) head part, as this is also round in analogue dermatoscopes. The first display preferably occupies a large portion of the surface of the head part, in particular at least 80%, preferably at least 90% of the surface of the head part. This enables a large display area and thus a large depiction of the skin section to be examined with a relatively compact size of the head part. This is also aesthetically pleasing.

According to one embodiment, the control unit is formed to display a live view of the skin section to be examined. The digital dermatoscope or its imaging unit can therefore, as already mentioned, preferably acquire multiple images per second and display them on the first display as a live view, i.e. as live video. For example, the digital dermatoscope can be formed to acquire at least 15 images per second, preferably 24 images per second, more preferably at least 30 images per second, more preferably at least 60 images per second.

This means that the digital dermatoscope can immediately display the skin to be examined, which is currently placed under the spacer attachment, without any time delay. This means that skin diseases or dermatoses of the skin to be examined can be detected quickly and easily. This also simulates an analogue dermatoscope. However, the digital dermatoscope makes it possible, for example, to store images or videos and also to display a still image of skin diseases or dermatoses over a longer period of time, even if the digital dermatoscope is no longer in contact with the skin.

According to one embodiment, the control unit is formed to enlarge an image of the skin section to be examined displayed on the first display by means of a digital zoom in response to an operating input. The digital dermatoscope can be formed to receive a corresponding operating input. For example, the digital dermatoscope can comprise an operating element for receiving an operating input to trigger a zoom function. Such an operating element can be provided by a further display, in particular by the second display arranged in the handle part, which will be explained later. Preferably, however, the first display is formed as a touch display and is formed to receive such an operating input, as already mentioned. In particular, the digital dermatoscope can be formed to receive such an operating input during the live view of the skin on the first display, so that the image can be zoomed in or out “live”. This type of operation is particularly intuitive. The digital zoom makes it possible to enlarge (or minimise) the skin section to be examined during the examination. This allows the operator to see details that might not be recognisable with a conventional analogue dermatoscope. Nevertheless, an advantageous fixed focal length can be maintained.

According to one embodiment, the first display is formed to display the image acquired by the imaging unit in other colours, in particular in false colours or in black and white. The digitalisation of the dermatoscope according to the invention enables the skin to be displayed not only in true colour, but also distorted or in black and white, which can particularly emphasise some skin characteristics. This makes it easier to recognise any skin diseases or dermatoses of the skin.

According to one embodiment, the digital dermatoscope comprises a second display in the form of a touch display arranged on an upper side of the handle part for displaying operating elements for operating the digital dermatoscope. The second display also is an electronic display. The second display is formed to display one or more operating elements, for example an operating element for executing the zoom function mentioned above.

Preferably, the second display is formed to display one or more of the following operating elements: an operating element for displaying the image acquired by the imaging unit on the first display in other colours, in particular in false colours or black and white, an operating element for changing the contrast of the image displayed on the first display, an operating element for displaying previously acquired images. The second display can also be formed to display status information of the digital dermatoscope, for example to display the current battery state/charging state of a battery of the digital dermatoscope, to display the current time and/or to display the reception quality of a WLAN signal (WLAN=Wireless Local Area Network). A second display of this kind can be used to operate the plurality of novel functions of the digital dermatoscope in a simple and intuitive way. To protect the second display, a cover glass can be arranged above the second display. In particular, this can be the same cover glass that is also provided for the first display.

According to one embodiment, the second display has a shape corresponding to the shape of the handle part. In particular, the handle part and/or the second display has a rectangular shape. In particular, the second display and the handle part have corresponding rectangular shapes. In particular, a rectangular shape of the handle part here means a flat, rectangular surface of the handle part. Although the rectangular shape of the handle part differs from the usually cylindrical handle parts of known analogue dermatoscopes, it allows efficient operation of the digital dermatoscope via the second display. The second display can, for example, extend over at least 40% of the upper side of the handle part, preferably over at least 50%, particularly preferably over at least 60%. This allows multiple operating elements to be displayed at the same time and while there is enough space to grasp the handle part with the hand without accidentally operating one of the operating elements.

According to one embodiment, the digital dermatoscope has a housing frame enclosing the handle part and the head part. In one embodiment, the housing frame consists of aluminium, in particular of machined aluminium. According to one embodiment, the imaging unit housing consists of aluminium, in particular of machined aluminium. Preferably, the housing frame and/or the imaging unit housing is machined in one piece from aluminium. More preferably, the aluminium housing frame and/or the aluminium imaging unit housing has a glass-bead blasted and/or anodised surface. More preferable, the aluminium imaging unit housing has a glass-bead blasted and/or anodised surface. In a further embodiment, the housing frame is formed as a heat sink for the electronic components contained therein. In particular, the housing frame forms such a heat sink if it consists of aluminium, as aluminium has a relatively high thermal conductivity. Such a housing frame or such an imaging unit housing is particularly stable and at the same time requires little material. Additionally, aluminium is particularly aesthetically pleasing and resistant to environmental influences, especially when formed integrally and/or when it is glass-bead blasted/anodised. However, the housing frame and/or the imaging unit housing can also consist of magnesium, stainless steel or plastic.

According to one embodiment, the imaging unit has at least one polarisation filter. In particular, the imaging unit can have two polarisation filters, which are preferably arranged at 90° rotated to each other. A first polarisation filter can be provided for illumination and a second polarisation filter for image acquisition. Cross-polarised detection is therefore possible. Preferably, the imaging unit has illumination elements which are formed to emit both polarised light and non-polarised light. In particular, the digital dermatoscope is thus formed to acquire a first image with polarised light as well as a second image with non-polarised light. Comparing the second image with the first image allows conclusions to be drawn about possible skin diseases or dermatoses (differential diagnosis). This is of particular advantage with the digital dermatoscope according to the invention, as the two images can be recorded and subsequently analysed.

According to one embodiment, the digital dermatoscope comprises a cover glass pane arranged on an upper side of the digital dermatoscope and extending over the head part and the handle part. As already mentioned, a cover glass pane can be arranged above the first display and/or the second display to protect the displays. According to this embodiment, the cover glass pane extends over the upper side of the head part and handle part, preferably over essentially the entire surface. This provides good protection and is aesthetically pleasing.

According to one embodiment, the digital dermatoscope comprises an operating button arranged on a side of the handle for triggering a predefined operating function of the digital dermatoscope. In particular, the operating button is arranged in such a way that it can be operated by the thumb of the hand without changing the grasping position when grasping the handle part with the hand. The operating function can be, for example, switching on and/or switching off the digital dermatoscope.

According to one embodiment, the digital dermatoscope comprises a rechargeable battery arranged in the handle part for supplying power to the electronic components of the digital dermatoscope. The battery is preferably a lithium-ion battery, particularly preferably a lithium-polymer battery. Due to its solid polymer electrolyte, a lithium-polymer battery particularly allows one of space and weight, as it fits better into the handle part of the housing, for example as a “pouch bag”. The battery is preferably arranged to supply power to the control unit and the first display, and in particular also to supply power to the second display, a memory unit and the illumination unit. The battery can be recharged via a charging port in the digital dermatoscope. The charging port can, for example, comprise a charging port for a charging plug and/or a charging port for wireless charging.

According to one embodiment, the digital dermatoscope comprises a non-volatile memory unit for electronically storing images and/or videos acquired by the imaging unit.

According to one embodiment, the digital dermatoscope comprises a data transfer port, in particular a USB port, preferably arranged at one end of the handle part, for establishing a data connection between the digital dermatoscope and an electronic device, in particular a tablet, a computer and/or a smartphone. The data transfer port can also be used to charge a battery of the digital dermatoscope, i.e. it can also form the charging port already mentioned.

According to one embodiment, the digital dermatoscope comprises a communication unit for wireless communication with an electronic device, in particular a tablet, a computer and/or a smartphone. The communication unit can be formed for communication via WLAN and/or Bluetooth.

According to one embodiment, the imaging unit comprises an illumination unit with multiple illumination elements annularly arranged for illuminating the skin section to be examined, as already mentioned. Preferably, the illumination elements are LEDs. According to one embodiment, the illumination elements are arranged at an angle to the optical axis. According to one embodiment, the illumination elements are arranged so that their angle to the optical axis can be adjusted. According to one embodiment, the illumination elements comprise a first number of LEDs, for example six LEDs, for emitting polarised light or for generating polarised light on the skin section to be examined, and a second number of LEDs, for example six LEDs, for emitting non-polarised light. Preferably, the first and second numbers are identical. The LEDs can be arranged uniformly around the circumference of the ring, in particular alternating between one LED for emitting polarised light and one LED for emitting non-polarised light. This allows the differential diagnosis mentioned above to be carried out in a simple fashion, in particular without the need for a polarisation filter.

According to one embodiment, the spacer attachment is held magnetically on the imaging unit housing. The spacer attachment can be made of magnetic stainless steel, for example. The imaging unit housing can have one or more magnetic elements for magnetically holding the spacer attachment on the imaging unit housing. The magnetic elements are preferably arranged inside the imaging unit housing so that dust or other foreign objects cannot penetrate from the outside. Magnetically attaching the spacer attachment to the imaging unit housing in this way simplifies changing the spacer attachments.

According to one embodiment, the spacer attachment has an opening at an end thereof facing away from the imaging unit housing and contacting the skin to be examined during a skin examination, as already mentioned. According to one embodiment, the opening is closed with a cover glass. When examining the skin, an immersion liquid can first be applied to the cover glass and/or the skin section to be examined and then the spacer attachment with the cover glass can be brought into contact with the skin section to be examined. A contact dermatoscopy can therefore be performed. According to one embodiment, the opening of the spacer attachment is not closed, i.e. in particular has no cover glass. Such a spacer attachment is particularly suitable for the examination of raised skin lesions or blood vessels that would disappear under pressure. During contact dermatoscopy, the pressure on the examined lesions can lead to exsanguination of the vessels, which can make diagnosis more difficult. In this sense, a “non-contact” dermatoscopy can then be performed with the digital dermatoscope, wherein the opening edge of the spacer attachment circumscribing the opening is still in contact with the skin during the examination.

According to one embodiment, the digital dermatoscope is waterproof. Such waterproofness can be in accordance with the IPX standard and can be achieved, for example, via sealing rings, in particular via a sealing ring arranged between the imaging unit and the head part and/or a sealing ring arranged between the lens and the image sensor of the imaging unit. Any charging or data transfer port provided can, for example, be moulded into the handle part and thence be waterproof.

The above-mentioned embodiments can be combined with one another.

Example embodiments of the invention are explained hereinafter with reference to the figures. They show schematically:

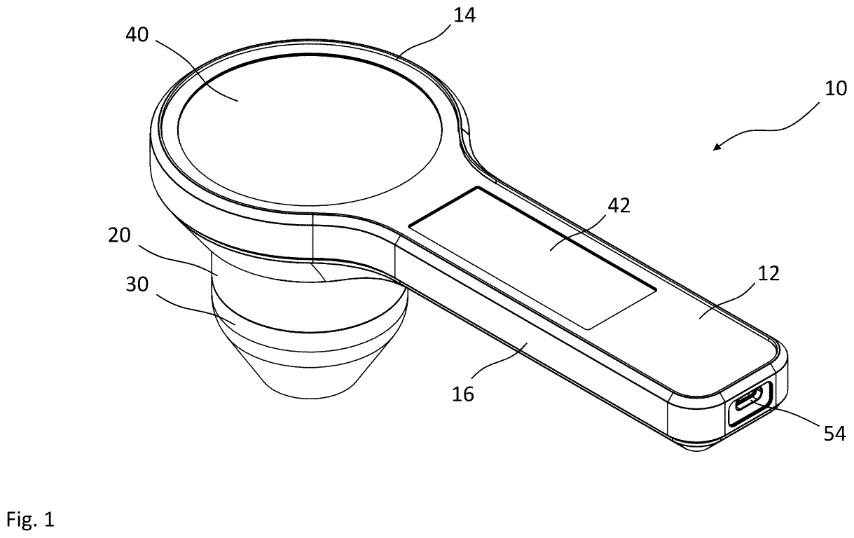

FIG. 1 a perspective view of the digital dermatoscope according to the invention,

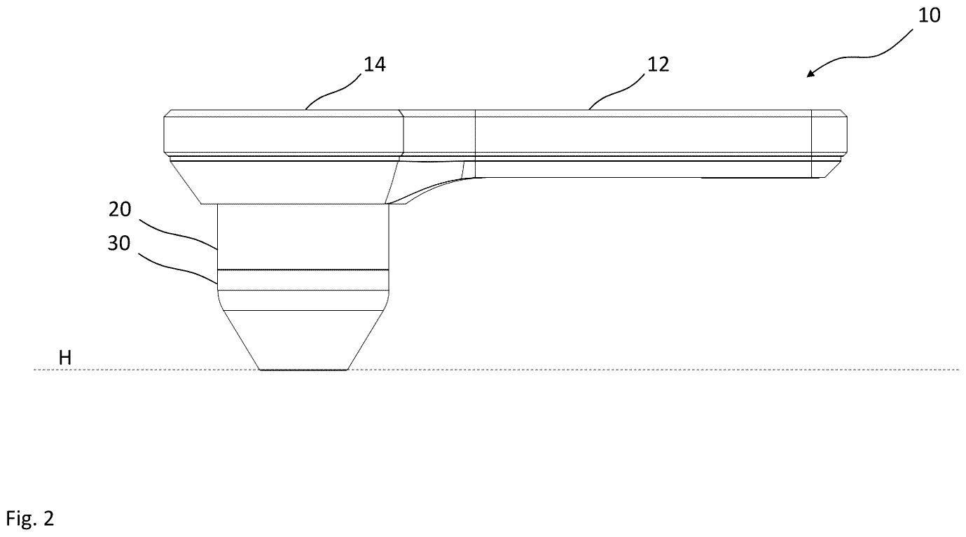

FIG. 2 a first side view of the digital dermatoscope of FIG. 1,

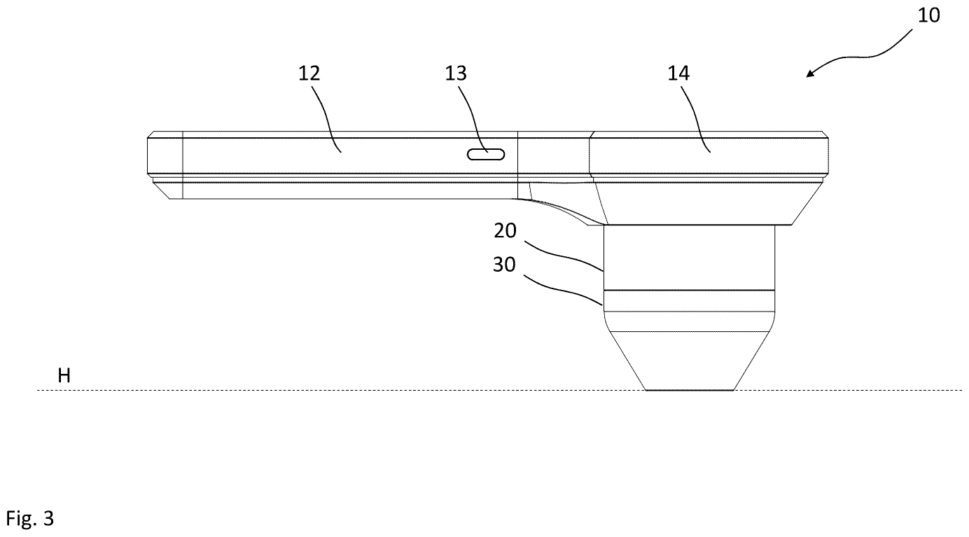

FIG. 3 a second side view of the digital dermatoscope of FIG. 1,

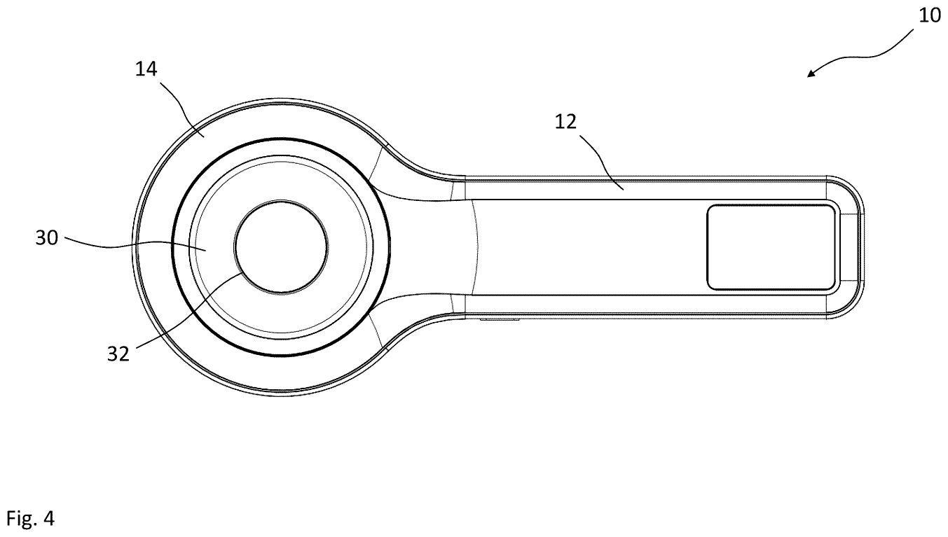

FIG. 4 a bottom view of the digital dermatoscope of FIG. 1,

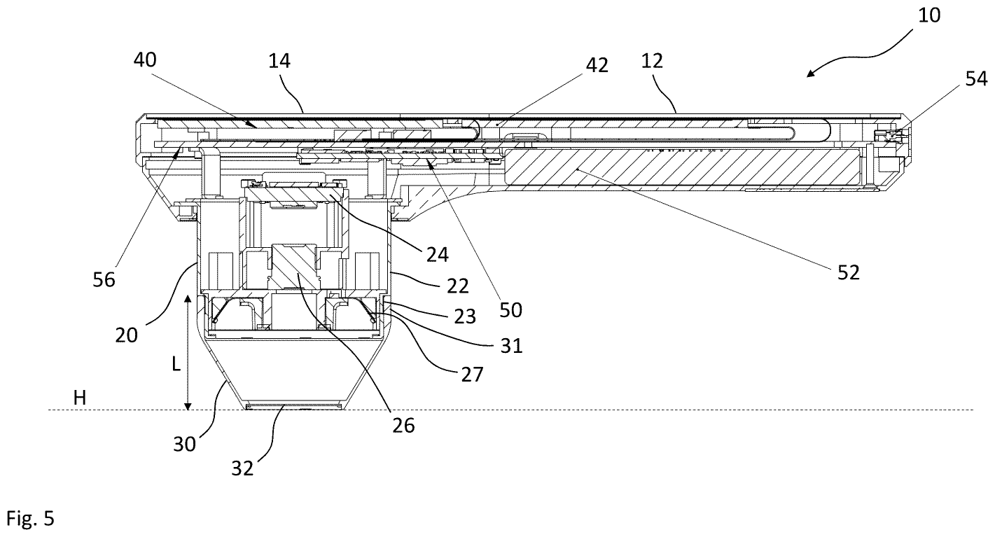

FIG. 5 a sectional view of the digital dermatoscope of FIG. 1,

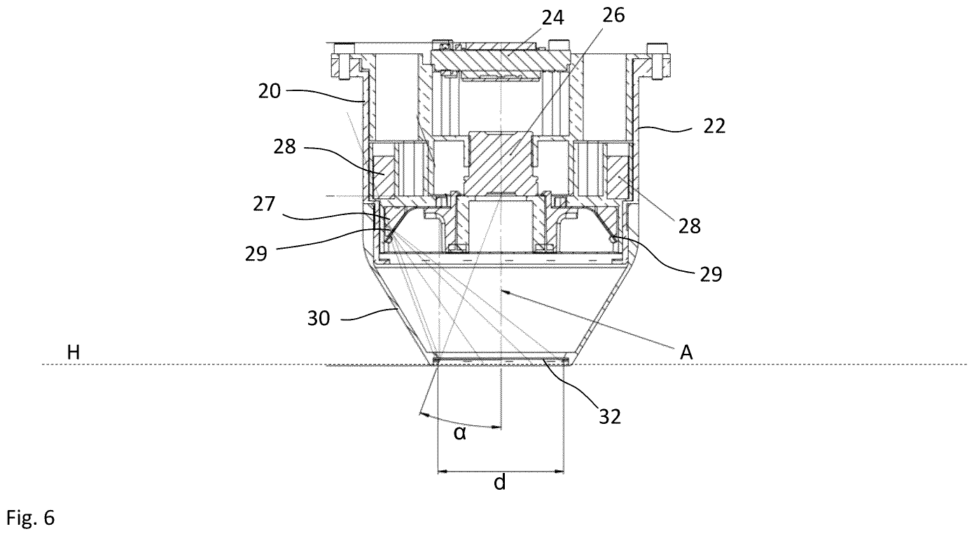

FIG. 6 a sectional view of the imaging unit of the digital dermatoscope of FIG. 1, and

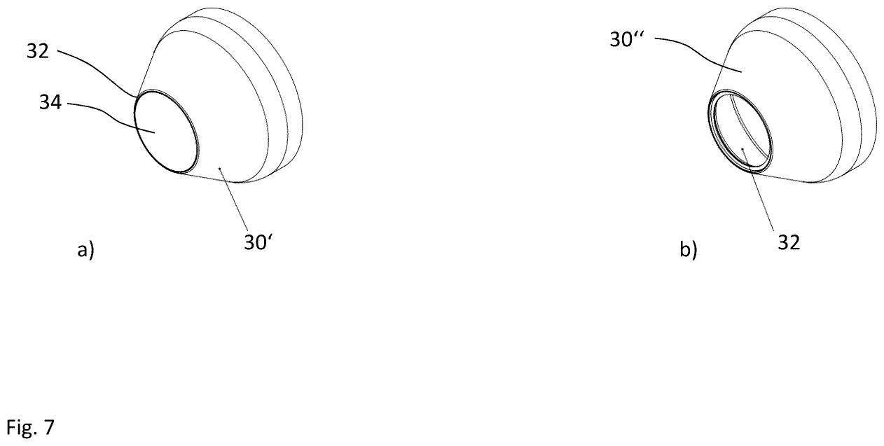

FIG. 7 two spacer attachments for the digital dermatoscope of FIG. 1.

Unless otherwise stated, the same reference signs are used below to denote the same objects. Terms such as “top” and “upper side” or “bottom” and “lower side” refer to a state of use of the digital dermatoscope in which the displays are facing the operator and can therefore be read by the operator and the spacer attachment is placed on the skin to be examined.

FIGS. 1 to 6 show a digital dermatoscope 10 according to the invention. The digital dermatoscope 10 comprises a handle part 12 that can be grasped by the hand of an operator and a head part 14 adjoining the handle part 12. The digital dermatoscope 10 is formed as a hand-held device, as an operator can pick it up (by grasping the handle part 12) and use it as intended to examine a patient's skin. The handle part 12 and the head part 14 are enclosed in a housing frame 16, which is preferably machined from aluminium.

The digital dermatoscope 10 further comprises an imaging unit 20, which is arranged on a lower side of the head part 14, as well as a spacer attachment 30, which is arranged in front of the imaging unit 20 and at the free end thereof. The imaging unit 20 has an imaging unit housing 22, an image sensor 24 accommodated in the imaging unit housing 22, a lens 26 with a fixed focal length, and an illumination unit 27 comprising an LED ring for illumination. The illumination unit 27 can, for example, have six LEDs for emitting polarised light and six LEDs for emitting non-polarised light, which are arranged uniformly and alternately around the circumference of the LED ring. The LEDs 29 are arranged at an angle to the optical axis A. The spacer attachment 30 has a length L matched to the imaging unit 20, in particular to the position of the lens 26. This ensures that the focal plane of the imaging unit 20 is situated in the area of an opening 32 of the spacer attachment 30. The circular opening 32 has an opening diameter d of 23.8 mm, for example. Due to the distance of the entrance of the lens 26 to the opening 32, an opening angle α is 20.40°, for example (see FIG. 6).

The spacer attachment 30 is detachably held on the imaging unit housing 22 by means of magnetic elements 28 arranged inside the imaging unit housing 22 (see FIG. 6). For this purpose, as can be seen in FIG. 5, for example, the imaging unit housing 22 has a circular recess 23 at the end thereof and the spacer attachment 30 has an endpiece 31 shaped to correspond to the circular recess 23, so that, when the spacer attachment 30 is attached to the imaging unit housing 22, the endpiece 31 overlaps with the circular recess 23. By overcoming the magnetic force, the spacer attachment 30 can be detached from the imaging unit housing 22 and replaced by a different spacer attachment 30, for example by a spacer attachment with a different opening diameter d of the opening 32.

A first display 40 is arranged in the head part 14 on the upper side thereof. The first display has a circular shape corresponding to the shape of the head 14, as can be seen in FIG. 1. In addition, a second display 42 is arranged on the upper side of the handle part 12, which has a rectangular shape corresponding to the shape of the handle part 12. In this example embodiment, the second display 42 extends over a large portion (more than 50%) of the upper side of the handle part 12. Both displays 40, 42 are touch displays.

The digital dermatoscope 10 further comprises internal components, including a control unit 50, a battery 52 installed in the handle part 12, a charging and data transfer port 54 installed at the end of the handle part 12, and a carrier board 56 connected to the control unit 50. In particular, the control unit 50 comprises a printed circuit board (PCB) with one or more processors. The control unit 50 is used to drive the electronic components of the digital dermatoscope 10. The digital dermatoscope 10 can also comprise a memory unit, in particular as part of the printed circuit board, for storing acquired images. The battery 12 is used to supply power to all electronic components and can be charged via the charging and data transfer port 54.

In the state of use of the digital dermatoscope 10, the spacer attachment 30 of the digital dermatoscope 10 is placed on a skin surface H to be examined. In doing so, the spacer attachment 30 contacts the skin to be examined at least with its opening edge circumscribing the opening 32. Thus, the opening 32 circumscribes a section of skin to be examined. The operator can switch the digital dermatoscope on and off, for example via an operating button 13 located on the side of the handle part 12. When switched on, the illumination unit 27 emits visible light through its LEDs 29 and thus illuminates the skin section to be examined, as can be seen particularly in FIG. 6. Due to the angled position of the LEDs 29 relative to the optical axis A, particularly good illumination of the section of skin to be examined is achieved. The illuminated skin reflects the light emitted by the illumination unit 27 and throws it into the lens 26, which focusses the received light onto the image sensor 24. Based on the signals received by the image sensor 24, which is for example a twelve-megapixel sensor, the control unit 50 creates a digital image of the skin section to be examined and displays this on the first display 40. The control unit 50 is thus formed (arranged, programmed) to drive the first display 40 for displaying images of a skin section to be examined acquired by means of the imaging unit 20.

Meanwhile, the second display 42 provides operating elements for operating the digital dermatoscope 10, for example an operating element for displaying the image acquired by the imaging unit on the first display in other colours, in particular in false colours or black and white, an operating element for changing the contrast of the image displayed on the first display, and/or an operating element for displaying previously acquired images. The operator can easily reach these operating elements with their thumb, for example, when they are holding the handle part 12 in their hand.

The first display 40 is situated in the optical axis A of the imaging unit 20, so that the optical axis A passes through the centre of the first display 40. This makes using the digital dermatoscope 40 particularly intuitive, as the operator has the impression of “looking through the dermatoscope” in a straight line, as with the analogue dermatoscopes they are already familiar with. The circular shape of the first display 40 also creates a similarity to an analogue dermatoscope, which contributes to intuitive operation. This also enables particularly effective skin examinations, as most skin diseases or dermatoses usually have an at least approximately round shape.

The first display 40 is formed as a touch display to recognise touch gestures performed by the operator using their fingers on the display surface and to perform an action in response to recognising a predefined touch gesture. Therefore, the display is preferably formed to recognise a touch gesture that triggers a zoom function. This allows the operator to enlarge or minimise, directly on the first display 40, an image of the skin section to be examined being displayed on the first display using a touch gesture, in particular by using a digital zoom. The first display 40 adopts the “magnifying glass character” of an analogue dermatoscope and transfers it into the digital world for the operator.

The digital dermatoscope according to the invention enables a simpler, more user-friendly examination of the skin as well as a more reliable diagnosis.

FIG. 7 shows two different spacer attachments 30′, 30″, which are suitable for use with the digital dermatoscope 10 according to the invention.

In the spacer attachment 30′ shown in FIG. 7a, the opening 32 is closed with a cover glass 34. When examining the skin, an immersion liquid is first applied to the cover glass 34 and/or the skin section to be examined and then the spacer attachment 30′ with the cover glass 34 are brought into contact with the skin section to be examined. A contact dermatoscopy can therefore be performed.

In the spacer attachment 30″ shown in FIG. 7b, the opening 32 is free, i.e. not closed with a cover glass. The spacer attachment 30 is particularly suitable for examining skin lesions that are raised above the surrounding skin surface or blood vessels that would disappear under pressure. In this sense, a “non-contact” dermatoscopy can then be performed with the digital dermatoscope 10, wherein the opening edge of the spacer attachment 30″ circumscribing the opening 32 is still in contact with the skin during the examination.

### LIST OF REFERENCE SIGNS

10 Digital dermatoscope
12 Handle part
13 Operating button
14 Head part
16 Housing frame
20 Imaging unit
22 Imaging unit housing
23 Recess
24 Image sensor
26 Lens
27 Illumination unit
28 Magnetic elements
29 LEDs
30, 30′, 30″ Spacer attachment
31 Endpiece
32 Opening
34 Cover glass
40 First display
42 Second display
50 Control unit
52 Battery
54 Charging and data transfer port
56 Carrier board
A Optical axis
d Diameter of the opening 32
H Skin surface

1. A digital dermatoscope for skin examination, the digital dermatoscope being formed as a hand-held device, wherein the digital dermatoscope comprises a handle part which can be grasped by a hand and a head part adjoining the handle part and having an imaging unit, wherein the imaging unit has an imaging unit housing arranged on a lower side of the head part, an image sensor accommodated inside the imaging unit housing, and a lens, wherein an exchangeable spacer attachment contacting the skin to be examined during a skin examination is arranged at an end of the imaging unit housing, the digital dermatoscope further comprising a control unit and a first display arranged on an upper side of the head part, wherein the control unit is formed to drive the first display to display images of a skin section to be examined acquired by means of the imaging unit, wherein the first display has a circular shape corresponding to the shape of the head part.

2. (canceled)

3. The digital dermatoscope of claim 1, wherein the control unit is formed to display a live view of the skin section to be examined on the first display.

4. The digital dermatoscope of claim 1, wherein the control unit is formed to enlarge an image of the skin section to be examined displayed on the first display by means of a digital zoom in response to an operating input.

5. The digital dermatoscope of claim 1, further comprising a second display arranged on an upper side of the handle part and formed as a touch display for displaying operating elements for operating the digital dermatoscope.

6. The digital dermatoscope of claim 1, wherein the digital dermatoscope has a housing frame enclosing the handle part and the head part, wherein the housing frame preferably consists of aluminium, in particular of machined aluminium.

7. The digital dermatoscope of claim 1, wherein the imaging unit housing consists of aluminium, in particular of machined aluminium.

8. The digital dermatoscope of claim 1, wherein the imaging unit comprises a polarisation filter.

9. The digital dermatoscope of claim 1, comprising a rechargeable battery, in particular a lithium-polymer battery, arranged in the handle part for supplying power to the electronic components of the digital dermatoscope, in particular to the control unit and the first display.

10. The digital dermatoscope of claim 1, comprising a non-volatile memory unit for electronically storing images and/or videos acquired by the imaging unit.

11. The digital dermatoscope of claim 1, comprising a port, preferably arranged at one end of the handle part, for charging a battery of the digital dermatoscope and/or for establishing a data connection between the digital dermatoscope and an electronic device, in particular a tablet.

12. The digital dermatoscope of claim 1, comprising a communication unit for wireless communication with an electronic device, in particular a tablet.

13. The digital dermatoscope of claim 1, wherein the imaging unit has an illumination unit with a plurality of annularly arranged illumination elements, in particular LEDs, for illuminating the skin section to be examined.

14. The digital dermatoscope of claim 13, wherein the illumination elements are arranged at an angle to an optical axis.

15. The digital dermatoscope of claim 1, wherein the spacer attachment is magnetically held to the imaging unit housing.

16. A digital dermatoscope for skin examination, the digital dermatoscope being formed as a hand-held device, wherein the digital dermatoscope comprises:
a handle part which can be grasped by a hand;
a head part adjoining the handle part and having an imaging unit, wherein the imaging unit includes:
an imaging unit housing arranged on a lower side of the head part,
an image sensor accommodated inside the imaging unit housing,
a lens, and
an exchangeable spacer attachment contacting the skin to be examined during a skin examination arranged at an end of the imaging unit housing,
a first display arranged on an upper side of the head part, wherein the first display has a circular shape corresponding to the shape of the head part; and
a control unit formed to drive the first display to display images of a skin section to be examined acquired by the imaging unit, the control unit formed to display a live view of the skin section to be examined on the first display.

17. The digital dermatoscope of claim 16, wherein the imaging unit has an illumination unit with a plurality of annularly arranged illumination elements, in particular LEDs, for illuminating the skin section to be examined.

18. The digital dermatoscope of claim 17, further comprising a second display arranged on an upper side of the handle part and formed as a touch display for displaying operating elements for operating the digital dermatoscope.

19. The digital dermatoscope of claim 18, wherein the imaging unit comprises a polarisation filter.

20. The digital dermatoscope of claim 19, wherein the illumination elements are arranged at an angle to an optical axis (A).

21. A digital dermatoscope for skin examination, the digital dermatoscope being formed as a hand-held device, wherein the digital dermatoscope comprises:
a handle part which can be grasped by a hand;
a head part adjoining the handle part and having an imaging unit, wherein the imaging unit includes:
an imaging unit housing arranged on a lower side of the head part and consisting of aluminium,
an image sensor accommodated inside the imaging unit housing,
an illumination unit with a plurality of annularly arranged illumination elements, in particular LEDs, for illuminating a skin section to be examined, wherein the illumination elements are arranged at an angle to an optical axis,
a polarisation filter,
a power source arranged in the handle part for supplying power to electronic components of the digital dermatoscope,
a lens, and
an exchangeable spacer attachment contacting the skin to be examined during a skin examination arranged at an end of the imaging unit housing,
a housing frame enclosing the handle part and the head part consisting of aluminium;
a first display arranged on an upper side of the head part and electrically connected to the power source, wherein the first display has a circular shape corresponding to the shape of the head part;
a second display arranged on an upper side of the handle part, electrically connected to the power source, and formed as a touch display for displaying operating elements for operating the digital dermatoscope; and
a control unit formed to drive the first display to display images of the skin section to be examined acquired by the imaging unit, the control unit formed to display a live view of the skin section to be examined on the first display, wherein the control unit is formed to enlarge an image of the skin section to be examined displayed on the first display by means of a digital zoom in response to an operating input.
